(19) United States
(12) Patent Application Publication
(10) Pub. No.: US 2022/0193324 A1
(43) Pub. Date: Jun. 23, 2022

(54) NEGATIVE-PRESSURE SOURCE WITH SERVICE TIMER

(71) Applicant: KCI Licensing, Inc., San Antonio, TX (US)

(72) Inventors: Christopher Brian LOCKE, Bournemouth (GB); Richard Daniel John COULTHARD, Verwood (GB)

(21) Appl. No.: 17/586,522

(22) Filed: Jan. 27, 2022

Related U.S. Application Data
(63) Continuation of application No. 16/322,404, filed on Jan. 31, 2019, now abandoned.
(60) Provisional application No. 62/371,117, filed on Aug. 4, 2016.

(51) Int. Cl. A61M 1/00

(52) U.S. Cl. CPC A61M 1/81; A61F 13/00068; A61M 1/90; A61M 1/67

(57) ABSTRACT

An apparatus for providing negative-pressure therapy may comprise a negative-pressure chamber and a pneumatically-actuated service timer, which can be used to indicate an expiration or other service condition of the apparatus. The apparatus may further comprise an actuator operable to engage a timer fluid with a migration medium. The timer fluid and the migration medium may be selected so that migration time through the migration medium corresponds to an expiration condition of the apparatus. The timer may also provide indicia of migration progress or termination.

### RELATED APPLICATIONS

This application is a continuation of U.S. patent application Ser. No. 16/322,404, filed Jan. 31, 2019, which is a U.S. National Stage Entry of PCT/US2017/045224, filed Aug. 3, 2017, which claims the benefit under 35 USC § 119(e), of the filing of U.S. Provisional Patent Application Ser. No. 62/371,117, entitled “Negative-Pressure Source With Service Timer” filed Aug. 4, 2016, which are incorporated herein by reference for all purposes.

### TECHNICAL FIELD

The invention set forth in the appended claims relates generally to tissue treatment systems and more particularly, but without limitation, to providing an indication when a negative-pressure source has reached an expiration or other service condition.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy and tissue interfaces are widely known, improvements to therapy systems, components, and processes may benefit healthcare providers and patients.

### BRIEF SUMMARY

New and useful systems, apparatuses, and methods for identifying an end of life of a reduce-pressure or negative-pressure pump are set forth in the appended claims. Illustrative embodiments are also provided to enable a person skilled in the art to make and use the claimed subject matter.

In some embodiments, an apparatus for providing negative-pressure therapy may comprise a negative-pressure chamber and a pneumatically-actuated service timer, which can be used to indicate an expiration or other service condition of the apparatus. For example, the timer may comprise a timer fluid and a migration medium through which the timer fluid may migrate at a predetermined rate. The apparatus may further comprise an actuator operable to engage the timer fluid with the migration medium. In some examples, the actuator may comprise a membrane and a sacrificial seal. The sacrificial seal may be disposed between the membrane and the migration medium. In some embodiments, the sacrificial seal may be coupled to or integral with the membrane. For example, the membrane may form a reservoir having a fluid outlet, which can be coupled to the migration medium. The sacrificial seal may be disposed between the fluid outlet and the migration medium to prevent fluid transfer between the reservoir and the migration medium. The timer fluid may be disposed in the reservoir, for example, wherein the membrane is adapted to collapse in response to a pressure differential across the membrane. Collapse of the membrane can sufficiently increase the pressure of the timer fluid in the reservoir to break the sacrificial seal and open the fluid outlet to the migration medium, allowing transfer of the timer fluid from the reservoir to the migration medium through the fluid outlet. The timer fluid and the migration medium may be selected so that migration time through the migration medium corresponds to an expiration condition of the apparatus. The timer may also provide indicia of migration progress or termination. For example, the migration medium may be substantially covered with one or more transparent regions through which the timer fluid may be observed as it migrates through the region. The migration medium or the transparent regions may additionally or alternatively have graduated markings indicative of progression through the migration medium in some embodiments. In yet other example embodiments, the timer fluid may change color as it migrates through the migration medium.

In some examples, the apparatus may be a negative-pressure pump comprising a charging chamber. A piston may be disposed in the pump and configured to reciprocate within the pump to expand and contract the charging chamber. The negative-pressure pump may additionally include an actuator configured to initiate a timer in response to a reduction of pressure in the charging chamber in some embodiments. For example, the actuator can be configured to initiate the timer in response to a differential pressure across the actuator generated by expansion of the charging chamber. In other examples, the actuator may be configured to initiate the timer in response to an engagement between the piston and the actuator.

In other examples, the negative-pressure pump may include a piston disposed in a piston chamber and configured to move along an axis of the piston chamber. The negative-pressure pump further includes a timer configured to be initiated in response to a differential pressure across the timer generated by the movement of the piston along the axis of the piston chamber. For example, the timer can be configured to be initiated in response to receiving an external compression force that is external to the timer.

In other examples, the apparatus may consist essentially of a service timer. In some embodiments, a service timer may comprise a housing, which may include a first vent and a second vent. The first vent may be configured to provide fluid communication between an interior of the housing and an ambient environment, for example. The second vent may be configured to provide fluid communication between the interior of the housing and a negative-pressure chamber. The timer may also include a flexible membrane disposed in the interior of the housing. The flexible membrane can form a fluid reservoir in some embodiments. The timer may further include a migration medium disposed in the interior of the housing and configured to receive fluid from the fluid reservoir in response to a compression of the flexible membrane.

Other examples of a timer may include a flexible membrane forming a fluid reservoir in fluid communication with a piston chamber of a negative-pressure pump. The timer may also include a migration medium configured to receive fluid from the fluid reservoir in response to a compression of the flexible membrane.

Other examples of an apparatus for negative-pressure therapy may include a negative-pressure chamber. The apparatus may also include a migration medium and a reservoir of a timer fluid. The reservoir is configured to transfer the timer fluid to the migration medium in response to a pressure differential between the negative-pressure chamber and an ambient pressure.

In more specific examples, the apparatus may be a negative-pressure pump, which can include a piston chamber. The negative-pressure pump also includes a piston disposed in the piston chamber and configured to move along an axis of the piston chamber. The negative-pressure pump further includes a timer configured to provide an indication of an end of useable life of the negative-pressure pump.

Methods of operating a negative-pressure pump are also provided. In some examples, the method may include actuating a piston through a piston chamber of the negative-pressure pump. The method can also include initiating a timer of the negative-pressure pump in response to activating negative pressure in the piston chamber.

Example methods of operating a timer of a negative-pressure pump are also provided. The methods may include applying a compression force to a flexible membrane to initiate the timer. A timer fluid may be communicated from a fluid reservoir in the flexible membrane through a migration medium in response to the compression force.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a simplified functional block diagram of an example embodiment of a therapy system that can provide negative-pressure therapy in accordance with this specification;

FIG. 2 is a front view of an example of a negative-pressure source that may be associated with some embodiments of the therapy system of FIG. 1;

FIG. 3 is a cross-sectional view of the negative-pressure source of FIG. 2;

FIGS. 4A and 4B are detailed views of example service timers that may be associated with the negative-pressure source of FIG. 3;

FIGS. 5A and 5B are detailed views of other example service timers that may be associated with the negative-pressure source of FIG. 3;

FIGS. 6A and 6B are detailed views of other example service timers that may be associated with the negative-pressure source of FIG. 3;

FIG. 7 is a schematic diagram illustrating example methods to operate a negative-pressure source in accordance with this specification.

### DESCRIPTION OF EXAMPLE EMBODIMENTS

The following description of example embodiments provides information that enables a person skilled in the art to make and use the subject matter set forth in the appended claims, but may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

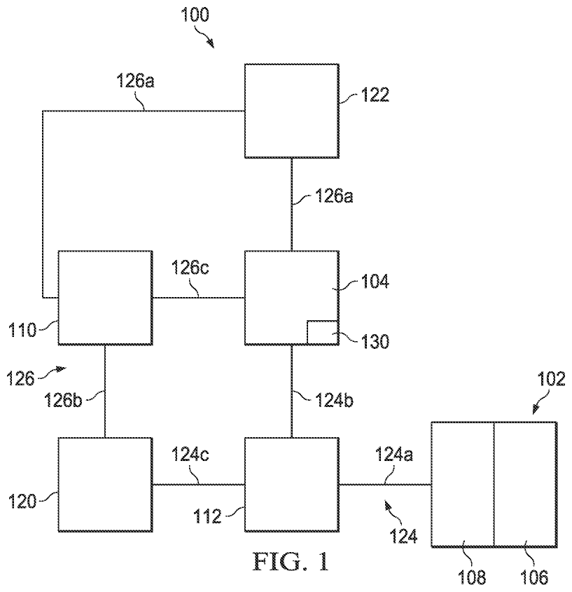

FIG. 1 is a simplified functional block diagram of an example embodiment of a therapy system 100 that can provide negative-pressure therapy to a tissue site in accordance with this specification.

The term “tissue site” in this context broadly refers to a wound, defect, or other treatment target located on or within tissue, including but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include negative-pressure supply, and may include or be configured to be coupled to a distribution component, such as a dressing. In general, a distribution component may refer to any complementary or ancillary component configured to be fluidly coupled to a negative-pressure supply in a fluid path between a negative-pressure supply and a tissue site. A distribution component is preferably detachable, and may be disposable, reusable, or recyclable. For example, a dressing 102 may be fluidly coupled to a negative-pressure source 104, as illustrated in FIG. 1. A dressing may include a cover, a tissue interface, or both in some embodiments. The dressing 102, for example, may include a cover 106 and a tissue interface 108. A regulator or a controller, such as a controller 110, may also be coupled to the negative-pressure source 104. A service timer may be coupled to or integral with some embodiments of the negative-pressure source 104. For example, a timer 130 can be configured to provide an indication of when a negative-pressure source 104 has reached an end to its service life or other expiration condition. It should be understood that an end of a negative-pressure source's service life includes when a negative-pressure source has a relatively high probability of less than optimal use, a relatively high probability of failure, a relatively high probability of becoming contaminated, or the like.

In some embodiments, a dressing interface may facilitate coupling the negative-pressure source 104 to the dressing 102. For example, such a dressing interface may be a T.R.A.C.® Pad or Sensa T.R.A.C.® Pad available from KCI of San Antonio, Tex. The therapy system 100 may optionally include a fluid container, such as a container 112, coupled to the dressing 102 and to the negative-pressure source 104.

Additionally, the therapy system 100 may include sensors to measure operating parameters and provide feedback signals to the controller 110 indicative of the operating parameters. As illustrated in FIG. 1, for example, the therapy system 100 may include a pressure sensor 120, an electric sensor 122, or both, coupled to the controller 110 via an electric conductor 126. The pressure sensor 120 may also be coupled or configured to be coupled to a distribution component and to the negative-pressure source 104 via an electric conductor 126. For example, as shown in FIG. 1, the electric conductor 126a provides electric communication between the electric sensor 122 and the controller 110; the electric conductor 126b provides electric communication between the pressure sensor 120 and the controller 110; the electric conductor 126c provides electric communication between the controller 110 and the negative-pressure source 104; and the electric conductor 126d provides electric communication between the electric sensor 122 and the negative-pressure source 104.

Components may be fluidly coupled to each other to provide a path for transferring fluids (such as at least one of liquid or gas) between the components. Components may be fluidly coupled through a fluid conductor 124, such as a tube. For example, fluid connector 124a provides fluid communication between the dressing 102 and the container 112; fluid connector 124b provides fluid communication between the negative-pressure source 104 and the container 112; and fluid conductor 124c provides fluid communication between the pressure sensor 120 and the container 112. A “tube,” as used herein, broadly includes a tube, pipe, hose, conduit, or other structure with one or more lumina adapted to convey a fluid between two ends. Typically, a tube is an elongated, cylindrical structure with some flexibility, but the geometry and rigidity may vary. In some embodiments, components may also be coupled by virtue of physical proximity, being integral to a single structure, or being formed from the same piece of material. Moreover, some fluid conductors 124 may be molded into or otherwise integrally combined with other components. Coupling may also include mechanical, thermal, electrical, or chemical coupling (such as a chemical bond) in some contexts. For example, a tube may mechanically and fluidly couple the dressing 102 to the container 112 in some embodiments.

In general, components of the therapy system 100 may be coupled directly or indirectly. For example, the negative-pressure source 104 may be directly coupled to the controller 110, and may be indirectly coupled to the dressing 102 through the container 112. The fluid mechanics of using a negative-pressure source to reduce pressure in another component or location, such as within a sealed therapeutic environment, can be mathematically complex. However, the basic principles of fluid mechanics applicable to negative-pressure therapy are generally well-known to those skilled in the art, and the process of reducing pressure may be described illustratively herein as “delivering,” “distributing,” or “generating” negative pressure, for example.

In general, exudates and other fluids flow toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications (such as by substituting a positive-pressure source for a negative-pressure source) and this descriptive convention should not be construed as a limiting convention.

“Negative pressure” generally refers to a pressure less than a local ambient pressure, such as the ambient pressure in a local environment external to a sealed therapeutic environment provided by the dressing 102. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. Similarly, references to increases in negative pressure typically refer to a decrease in absolute pressure, while decreases in negative pressure typically refer to an increase in absolute pressure. While the amount and nature of negative pressure applied to a tissue site may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −75 mm Hg (−9.9 kPa) and −300 mm Hg (−39.9 kPa).

A negative-pressure supply, such as the negative-pressure source 104, may be a reservoir of air at a negative pressure, or may be a manual or electrically-powered device that can reduce the pressure in a sealed volume, such as a vacuum pump, a suction pump, a wall suction port available at many healthcare facilities, or a micro-pump, for example. A negative-pressure supply may be housed within or used in conjunction with other components, such as sensors, processing units, alarm indicators, memory, databases, software, display devices, or user interfaces that further facilitate therapy. For example, in some embodiments, the negative-pressure source 104 may be combined with the controller 110 and other components into a therapy unit. A negative-pressure supply may also have one or more supply ports configured to facilitate coupling and de-coupling the negative-pressure supply to one or more distribution components.

The tissue interface 108 can be generally configured to contact a tissue site. The tissue interface 108 may be partially or fully in contact with the tissue site. If the tissue site is a wound, for example, the tissue interface 108 may partially or completely fill the wound, or may be placed over the wound. The tissue interface 108 may take many forms, and may have many sizes, shapes, or thicknesses depending on a variety of factors, such as the type of treatment being implemented or the nature and size of a tissue site. For example, the size and shape of the tissue interface 108 may be adapted to the contours of deep and irregular shaped tissue sites. Moreover, any or all of the surfaces of the tissue interface 108 may have projections or an uneven, course, or jagged profile that can induce strains and stresses on a tissue site, which can promote granulation at the tissue site.

In some embodiments, the tissue interface 108 may be a manifold. A “manifold” in this context generally includes any substance or structure providing a plurality of pathways adapted to collect or distribute fluid across a tissue site under pressure. For example, a manifold may be adapted to receive negative pressure from a source and distribute negative pressure through multiple apertures across a tissue site, which may have the effect of collecting fluid from across a tissue site and drawing the fluid toward the source. In some embodiments, the fluid path may be reversed or a secondary fluid path may be provided to facilitate delivering fluid across a tissue site.

In some illustrative embodiments, the pathways of a manifold may be interconnected to improve distribution or collection of fluids across a tissue site. In some illustrative embodiments, a manifold may be a porous foam material having interconnected cells or pores. For example, cellular foam, open-cell foam, reticulated foam, porous tissue collections, and other porous material such as gauze or felted mat generally include pores, edges, and/or walls adapted to form interconnected fluid channels. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

The average pore size of a foam may vary according to needs of a prescribed therapy. For example, in some embodiments, the tissue interface 108 may be a foam having pore sizes in a range of 400-600 microns. The tensile strength of the tissue interface 108 may also vary according to needs of a prescribed therapy. For example, the tensile strength of a foam may be increased for instillation of topical treatment solutions. In one non-limiting example, the tissue interface 108 may be an open-cell, reticulated polyurethane foam such as GranuFoam® dressing or VeraFlo® foam, both available from Kinetic Concepts, Inc. of San Antonio, Tex.

The tissue interface 108 may be either hydrophobic or hydrophilic. In an example in which the tissue interface 108 may be hydrophilic, the tissue interface 108 may also wick fluid away from a tissue site, while continuing to distribute negative pressure to the tissue site. The wicking properties of the tissue interface 108 may draw fluid away from a tissue site by capillary flow or other wicking mechanisms. An example of a hydrophilic foam is a polyvinyl alcohol, open-cell foam such as V.A.C. WhiteFoam® dressing available from Kinetic Concepts, Inc. of San Antonio, Tex. Other hydrophilic foams may include those made from polyether. Other foams that may exhibit hydrophilic characteristics include hydrophobic foams that have been treated or coated to provide hydrophilicity. In at least some embodiments, the tissue interface can also be a polymeric structure which is either compression or injection molded. In at least some embodiments, the tissue interface can be formed from a vacuum formed sheet of compatible hydrophobic material. One of ordinary skill in the art can identify the many types of tissue interfaces that can be implemented with the concepts of this disclosure.

The tissue interface 108 may further promote granulation at a tissue site when pressure within the sealed therapeutic environment is reduced. For example, any or all of the surfaces of the tissue interface 108 may have an uneven, coarse, or jagged profile that can induce microstrains and stresses at a tissue site if negative pressure is applied through the tissue interface 108.

In some embodiments, the tissue interface 108 may be constructed from bioresorbable materials. Suitable bioresorbable materials may include, without limitation, a polymeric blend of polylactic acid (PLA) and polyglycolic acid (PGA). The polymeric blend may also include without limitation polycarbonates, polyfumarates, and capralactones. The tissue interface 108 may further serve as a scaffold for new cell-growth, or a scaffold material may be used in conjunction with the tissue interface 108 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 106 may provide a bacterial barrier and protection from physical trauma. The cover 106 may also be constructed from a material that can reduce evaporative losses and provide a fluid seal between two components or two environments, such as between a therapeutic environment and a local external environment. The cover 106 may be, for example, an elastomeric film or membrane that can provide a seal adequate to maintain a negative pressure at a tissue site for a given negative-pressure source. The cover 106 may have a high moisture-vapor transmission rate (MVTR) in some applications. For example, the MVTR may be at least 300 g/m\hat{ }2 per twenty-four hours in some embodiments. In some example embodiments, the cover 106 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained.

An attachment device may be used to attach the cover 106 to an attachment surface, such as undamaged epidermis, a gasket, or another cover. The attachment device may take many forms. For example, an attachment device may be a medically-acceptable, pressure-sensitive adhesive that extends about a periphery, a portion, or an entire sealing member. In some embodiments, for example, some or all of the cover 106 may be coated with an acrylic adhesive having a coating weight between 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

A controller, such as the controller 110, may be a microprocessor or computer programmed to operate one or more components of the therapy system 100, such as the negative-pressure source 104. In some embodiments, for example, the controller 110 may be a microcontroller, which generally comprises an integrated circuit containing a processor core and a memory programmed to directly or indirectly control one or more operating parameters of the therapy system 100. Operating parameters may include the power applied to the negative-pressure source 104, the pressure generated by the negative-pressure source 104, or the pressure distributed to the tissue interface 108, for example. The controller 110 is also preferably configured to receive one or more input signals, such as a feedback signal, and programmed to modify one or more operating parameters based on the input signals.

Sensors, such as the pressure sensor 120 or the electric sensor 122, are generally known in the art as any apparatus operable to detect or measure a physical phenomenon or property, and generally provide a signal indicative of the phenomenon or property that is detected or measured. For example, the pressure sensor 120 and the electric sensor 122 may be configured to measure one or more operating parameters of the therapy system 100. In some embodiments, the pressure sensor 120 may be a transducer configured to measure pressure in a pneumatic pathway and convert the measurement to a signal indicative of the pressure measured. In some embodiments, for example, the pressure sensor 120 may be a piezoresistive strain gauge. The electric sensor 122 may optionally measure operating parameters of the negative-pressure source 104, such as the voltage or current, in some embodiments. Preferably, the signals from the pressure sensor 120 and the electric sensor 122 are suitable as an input signal to the controller 110, but some signal conditioning may be appropriate in some embodiments. For example, the signal may need to be filtered or amplified before it can be processed by the controller 110. Typically, the signal is an electrical signal, but may be represented in other forms, such as an optical signal.

The container 112 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

In operation, the tissue interface 108 may be placed within, over, on, or otherwise proximate to a tissue site. The cover 106 may be placed over the tissue interface 108 and sealed to an attachment surface near the tissue site. For example, the cover 106 may be sealed to undamaged epidermis peripheral to a tissue site. Thus, the dressing 102 can provide a sealed therapeutic environment proximate to a tissue site, substantially isolated from the external environment, and the negative-pressure source 104 can reduce the pressure in the sealed therapeutic environment. Negative pressure applied across the tissue site through the tissue interface 108 in the sealed therapeutic environment can induce macrostrain and microstrain in the tissue site, as well as remove exudates and other fluids from the tissue site, which can be collected in container 112.

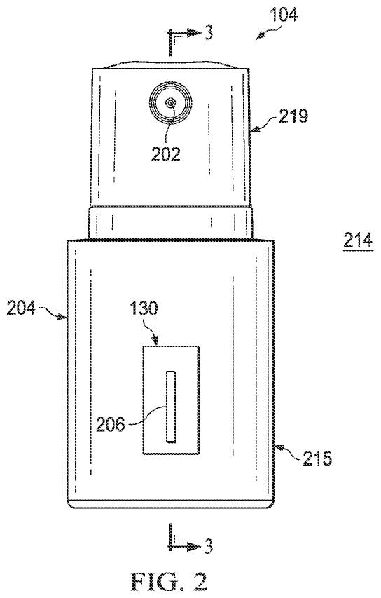

FIG. 2 is a front view of an example of the negative-pressure source 104, illustrating additional details that may be associated with some embodiments. In the example embodiment of FIG. 2, the negative-pressure source 104 comprises or consists essentially of a manually-actuated negative-pressure pump, which may include a negative-pressure outlet 202, a housing 204, a first barrel 215, and a second barrel 219. The timer 130 may be coupled to the housing 204 in some embodiments, and may include a view port 206. The negative-pressure outlet 202 is configured to transfer negative pressure to a dressing or other distribution component, for example via one or more conduits or connectors. For example, with reference to FIG. 1, the negative-pressure outlet 202 can be coupled to fluid connector 124b to provide negative-pressure to the dressing 102 via the container 112 and the fluid connector 124a.

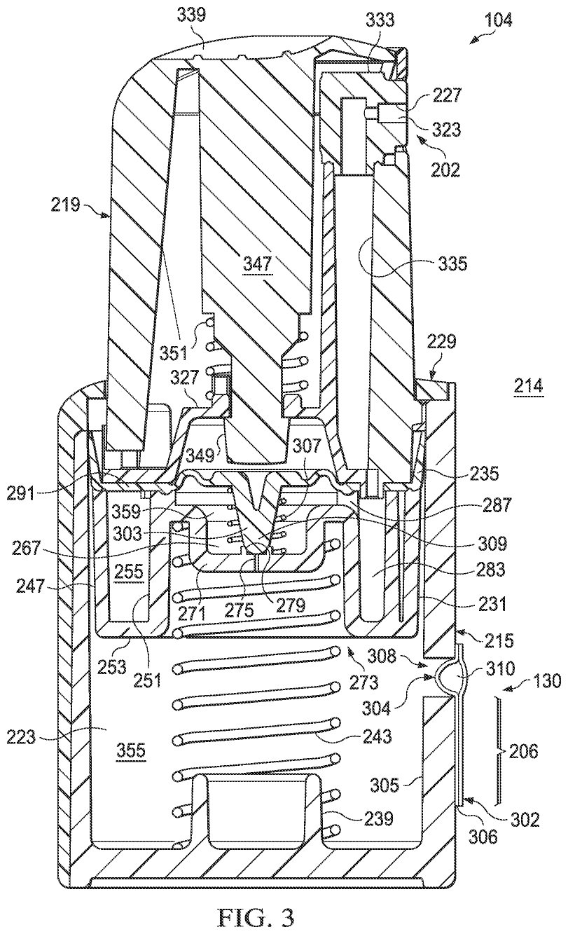

FIG. 3 is a cross-sectional view of the negative-pressure source 104 of FIG. 2 taken along section line 3-3. As shown in the example embodiment of FIG. 3, the negative-pressure source 104 includes a barrel ring 229, a piston 231, and a seal 235. The barrel ring 229 can be positioned at the open end of the first barrel 215 to circumscribe the second barrel 219. The barrel ring 229 can substantially reduce or eliminate large gaps between the first barrel 215 and the second barrel 219. When the negative-pressure source 104 is assembled as shown in the example of FIG. 3, the second barrel 219 may be slidingly received within the first barrel 215, defining a piston chamber 223. The piston 231 and seal 235 may be slidingly received within the piston chamber 223. Both the piston 231 and the seal 235 can be positioned in the piston chamber 223 between the second barrel 219 and the closed end of the first barrel 215, the seal 235 being positioned between the second barrel 219 and the piston 231.

The first barrel 215 may include a protrusion 239 extending from the closed end of the first barrel 215 into the piston chamber 223. A piston spring 243 or other biasing member can be positioned within the piston chamber 223 and received at one end of the piston spring 243 by the protrusion 239. The protrusion 239 can reduce lateral movement of the piston spring 243 within the piston chamber 223. A second end of the piston spring 243 can be disposed against the piston 231. The piston spring 243 generally biases the piston 231, the seal 235, and the second barrel 219 toward an extended position.

In some example embodiments, the piston 231 may include an outer wall 247 and an inner wall 251 joined by an outer floor 253. An annulus 255 can be defined between the outer wall 247 and the inner wall 251, and a plurality of radial supports can be positioned between the outer wall 247 and the inner wall 251 in the annulus 255. The radial supports can provide additional rigidity to the piston 231, yet the presence of the annulus 255 as well as the sizes and spacing of the radial supports within the annulus 255 can reduce the weight of the piston 231 as compared to a single-wall piston that includes no annulus. However, it should be apparent that either piston design would be suitable for the reduced pressure source described herein.

The piston 231 may further include an inner bowl 267 in some embodiments, which can be defined by the inner wall 251 and an inner floor 271. In one embodiment, the inner floor 271 may be two-tiered or multi-tiered, but the inner floor 271 may instead be single-tiered and/or substantially planar. The inner floor 271 may be positioned such that a recess 273 is defined beneath the inner floor 271 to receive an end of the piston spring 243. As illustrated in the example of FIG. 3, a regulator passage 275 can pass through the inner floor 271. A valve seat 279 may be positioned in the inner bowl 267 near the regulator passage 275, such that fluid communication through the regulator passage 275 may be controlled by selective engagement of the valve seat 279 with a valve body, such as a valve body 303. A well 283 may be positioned in the annulus 255 of the piston 231, and a channel 287 can fluidly connect the well 283 and the inner bowl 267. The channel 287 allows fluid communication between the well 283 and the inner bowl 267.

The valve body 303 can be positioned on the central portion 291 of the seal 235 in some example embodiments. Although valve bodies of many types, shapes and sizes may be used, the valve body 303 may be cone-shaped with an apex 309 that is adapted to sealingly engage the valve seat 279 of the piston 231. While the valve body 303 can be an integral part of the seal 235. The valve body 303 may alternatively be a separate component from the seal 235 that is provided to engage the valve seat 279.

In some embodiments, both the seal 235 and the valve body 303 can be made from an elastomeric material, which could include without limitation a medical grade silicone. While many different materials may be used to construct, form, or otherwise create the seal 235 and valve body 303, it is preferred that a flexible material be used to improve the sealing properties of the skirt portion 295 with the inner surface and the valve body 303 with the valve seat 279.

A spring 307 can bias the valve body 303 away from the piston 231 and the valve seat 279. One end of the spring 307 may be positioned concentrically around the valve seat 279 within the inner bowl 267 of the piston 231, while another end of the spring 307 may be positioned around the valve body 303. The biasing force provided by the spring 307 can urge the valve body 303 toward an open position in which fluid communication is permitted through the regulator passage 275. In one embodiment, when the spring 307 biases the valve body 303 toward the open position, only the central portion 291 of the seal 235 moves upward due to the flexibility of the seal. In another embodiment, the biasing force of the spring 307 may move the entire seal 235 toward the open position.

The second barrel 219 may include a boss 333. The boss 333 in the example of FIG. 3 includes the outlet port 227, which can be physically aligned with the aperture 323 to allow a delivery tube to be fluidly connected to the negative-pressure outlet 202. In one embodiment, the boss 333 is a ninety degree fluid fitting that permits the negative-pressure outlet 202 to fluidly communicate with a conduit.

The second barrel 219 may include an end cap 339 and a shaft 347. The shaft 347 may extend from the end cap 339, and can include an engagement end 349 opposite the end cap 339. If the second barrel 219 is assembled as shown in the example of FIG. 3, the shaft 347 may be substantially coaxial to a longitudinal axis of the second barrel 219 and extend through the passage in the floor 327 of the second barrel 219. A spring 351 can be positioned within the second barrel 219 such that one end of the spring 351 bears upon the floor 327 and another end of the spring 351 bears upon the shaft 347 or another portion of the second barrel 219. The spring 351 can bias the shaft 347 and other portions of the second barrel 219 toward a disengaged position in which the engagement end 349 of the shaft 347 does not bear upon the seal 235 or valve body 303. If a force is exerted on the second barrel 219, the engagement end 345 of the shaft 347 bears upon the seal 235 above the valve body 303, which forces the valve body 303 against the valve seat 279, thereby preventing fluid communication through the regulator passage 275.

In some embodiments, the negative-pressure source 104 may include a charging chamber 355. For example, if the negative-pressure source 104 is assembled as illustrated in FIG. 3, the charging chamber 355 may be defined within the first barrel 215 beneath the piston 231. The negative-pressure source 104 may also include a regulated chamber 359 in some embodiments. The regulated chamber 359 can be defined within the inner bowl 267 of the piston 231 beneath the seal 235, for example. The regulator passage 275 can allow selective fluid communication between the charging chamber 355 and the regulated chamber 359, depending on the position of the valve body 303. The regulated chamber 359 of FIG. 3 can fluidly communicate with the well 283 of the piston 231 through the channel 287. The well 283 can be aligned with a communication aperture of the seal 235 and the communication aperture of the second barrel 219, which allows fluid communication between the well 283 and the conduit 335 and negative-pressure outlet 202 of the second barrel 219. While the regulator passage 275 can be disposed within the piston 231, the regulator passage 275 could instead be routed through other components, such as the wall of the first barrel 215, for example. The regulator passage 275 could be any conduit that is suitable for allowing fluid communication between the chambers.

The view port 206 is configured to permit a viewing of a fluid passage so that timer fluid within the fluid passage can be viewed from a position from the ambient environment 214. The view port 206 is disposed on at least a part of a housing of the negative-pressure source 104. For example, the view port 206 can be disposed at least over a portion of the fluid passage that is located at the predetermined distance from the fluid reservoir, to be discussed herein. When fluid reaches the predetermined distance through the fluid passage from the fluid reservoir, the timer 130 can provide a viewable indication, via the view port 206, indicating that the negative-pressure source 104 has reached at least one of an expiration of useable life or useful life. In another example, the view port 206 can be disposed on another portion of the housing of the negative-pressure source 104 providing a viewing over a distance of the fluid passage. The view port 206 can be disposed over the fluid passage from the fluid reservoir to the predetermined distance within the fluid passage to provide a viewable indication of how close the negative-pressure source 104 is to reaching an expiration of at least one of useable life or useful life. The view port 206 can also be disposed over the fluid passage from an initial distance away from the fluid reservoir to the predetermined distance from the fluid reservoir to provide a viewable indication of how close the negative-pressure source 104 is to reaching an expiration of useable life.

In operation, the negative-pressure outlet 202 of the negative-pressure source 104 may be connected to a delivery tube or other conduit that is fluidly connected to a tissue site. Although a fluid canister could be integrated into the negative-pressure source 104, in some embodiments the negative-pressure source 104 is not intended to collect wound exudates or other fluids within any internal chamber. In some embodiments, the negative-pressure source 104 may either be used with low-exudating wounds, or an alternative collection system such as an external canister or absorptive dressing may be used to collect fluids.

The negative-pressure source 104 may be charged to increase negative-pressure in the charging chamber 355. For example, the piston 231 can be biased by the piston spring 243 toward a resting position where the piston chamber is at a maximum volume. The negative-pressure source 104 may be primed by compressing the second barrel 219 within the first barrel 215 to decrease the working volume of the charging chamber 355, and then releasing the second barrel 219 to allow the piston spring 243 to increase the working volume of the charging chamber 355.

In some embodiments, if the piston 231 is driven or moved from the resting position, fluid (such as air) can be pushed or forced out of the charging chamber 355 through the seal 235 into the ambient environment 214. If the piston 231 is released after being charged, the piston spring 243 can move the piston 231 toward the resting position, increasing the volume of the charging chamber 355. As the piston 231 returns to the resting position, the volume of the charging chamber 355 increases while the seal 325 prevents fluid from entering the charging chamber 355 from the ambient environment 214, which decreases pressure within the charging chamber 355. Accordingly, the negative-pressure source 104 can provide negative pressure to a dressing 102, for example, at a tissue site via the negative-pressure outlet 202.

As the negative-pressure source 104 is being charged by one or more compressions, air and other positively-pressurized gases should be expelled from the charging chamber 355 without entering the regulated chamber 359, which could counteract the negative pressure applied to the tissue site. To prevent positively pressurized gas from entering the regulated chamber 359, the shaft 347 can engage the seal 235 and valve body 303. As the second barrel 219 is compressed within the first barrel 215, the shaft 347 exerts a force on the valve body 303 that holds the valve body 303 in the closed position. Since the shaft 347 remains engaged during the entire compression, or charging stroke of the negative-pressure source 104, the air within the charging chamber 355 is vented past the seal 235 and not into the regulated chamber 359.

The regulated chamber 359 can be used to provide a desired therapy pressure that can be delivered to the outlet port 227 and the tissue site. For example, if the negative pressure in the regulated chamber 359 is less than the desired therapy pressure, the spring 307 can exert an upward force on the seal 235 that exceeds the force of the pressure differential exerted downward on the seal 235, and move the valve body 303 into an open position allowing fluid communication between the charging chamber 355 and the regulated chamber 359. If the negative pressure in the charging chamber 355 is greater than the negative pressure in the regulated chamber 359, negative pressure can be distributed from the charging chamber 355 to the regulated chamber 359 until the negative pressure in the regulated chamber 359, balanced against the atmospheric pressure above the seal 235, is sufficient to counteract the biasing force of the spring 307 and move the valve body 303 into the closed position. When the regulated chamber 359 is charged with the desired therapy pressure, this pressure may be delivered to the negative-pressure outlet 202.

While the negative-pressure source 104, including the first barrel 215, the second barrel 219, the piston 231, and the seal 235, have been described herein as being cylindrical, it will be readily apparent that all of these components may be any size or shape. Additionally, the relative positions of the valve seat 279 and the valve body 303 may be reversed such that the valve body 303 is positioned below the valve seat 279.

The timer 130 can be activated by movement of the piston 231. The timer 130 can be activated in response to a differential pressure across the timer 130, for example, which can be generated by increasing negative pressure in the charging chamber 355. In other examples, the timer 130 can be activated by an engagement between the piston 231 and the timer 130.

In the example embodiment of FIG. 3, the timer 130 includes a housing 302, an actuator 304, and a migration medium 306. The actuator 304 can be fluidly coupled to the charging chamber 355 on a first side. In some embodiments, the actuator 304 can extend through the aperture 308 and into the charging chamber 355. The housing 302 can be coupled to an exterior surface of the second barrel 219 in some embodiments. For example, the housing 302 can be configured to fit over an aperture 308, providing a seal over the aperture 308 and preventing fluid communication between the charging chamber 355 and the ambient environment 214. The actuator 304 can be disposed between the charging chamber 355 and the ambient environment 214. For example, the actuator 304 can be placed proximate to, over, in, or through the aperture 308. In some embodiments, the actuator 304 can form a fluid reservoir 310. The fluid reservoir 310 may contain a timer fluid, such as a colored fluid, configured to move through the migration medium 306. If the fluid reservoir 310 is compressed, timer fluid in the fluid reservoir 310 can be transferred to the migration medium 306. For example, compression of the fluid reservoir 310 may be caused by contact between the piston 231 and the actuator 304, or by a pressure differential across the actuator 304.

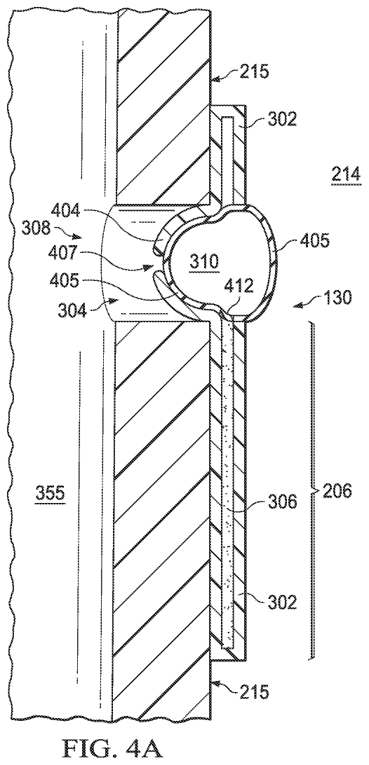

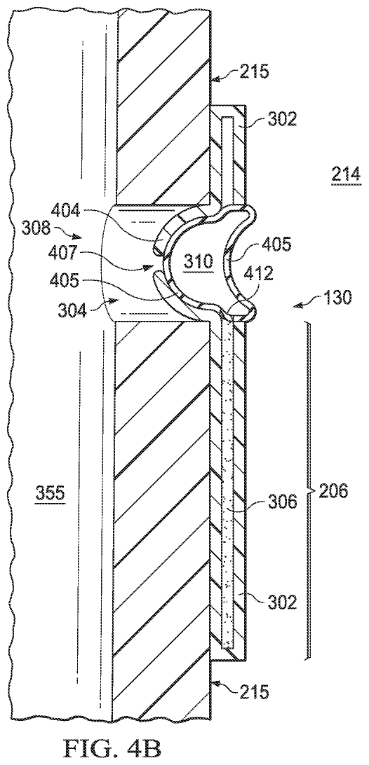

FIGS. 4A and 4B are schematic diagrams illustrating additional details that may be associated with some example embodiments of the timer 130 of FIG. 3. In the example embodiment of FIGS. 4A and 4B, the actuator 304 may include a shell 404 and a flexible membrane 405 with a sacrificial seal, such as a migration medium membrane 412. The shell 404 preferably includes a vent or other aperture, such as an aperture 407, adapted to allow fluid communication between the charging chamber 355 and the flexible membrane 405. While FIGS. 4A and 4B illustrate that the shell 404 is part of the housing 302, in some embodiments, the shell 404 may be a part of the charging chamber 355 or the aperture 308. FIG. 4A illustrates an example of the flexible membrane 405 in a non-deflected state. When the piston 231 is driven or moved through the charging chamber 355 and subsequently permitted to return to a resting position or resting state, the piston 231 generates or creates negative pressure in the charging chamber 355 and through the aperture 407 (such as a vent) of the shell 404 to the flexible membrane 405. The differential between the ambient pressure and the negative pressure in the charging chamber can deflect the flexible membrane 405 that is exposed to the ambient environment 214, compressing the fluid reservoir 310. The fluid reservoir 310 may also be compressed against shell 404. FIG. 4B illustrates an example of the flexible membrane 405 and the fluid reservoir 310 in a compressed state.

The deflection of the flexible membrane 405 and contact with the shell 404 can create a compression force on the fluid reservoir 310 and increase pressure in the fluid reservoir 310. If the pressure exceeds a predetermined threshold, timer fluid in the fluid reservoir 310 can break the migration medium membrane 412, allowing the timer fluid to move into the migration medium 306. Fluid moving into and through the migration medium 306 can provide an indication of a time relative to an initial movement of the piston 231 through the charging chamber 355. Thus, in some embodiments, migration of fluid through the migration medium 306 indicates a time. When the fluid reaches a distance (such as a predetermined distance) in the migration medium 306 from the migration medium membrane 412 or the fluid reservoir 310, the timer 130 provides an indication that the negative-pressure source 104 has reached a time when at least one of a usable life or a useful life of the negative-pressure source 104 is expired.

The migration medium membrane 412 can be configured to prevent fluid communication from the fluid reservoir 310 to the migration medium 306. In some embodiments, the migration medium membrane 412 can be a breakable membrane that is configured to break when the fluid reservoir is compressed sufficiently and allow fluid to communicate from the fluid reservoir 310 to the migration medium 306. For example, if negative pressure in the charging chamber 355 is increased to an operational negative-pressure, the pressure differential across the actuator 304 can compress the fluid reservoir 310 (and increase the pressure of the fluid in the fluid reservoir 310) sufficiently to rupture the migration medium membrane 412 and move the fluid to the migration medium 306. Fluid, such as timer fluid, can move through the migration medium 306 until it reaches a distance from the fluid reservoir 310 indicating that the negative-pressure source 104 has reached an expiration of at least one of useable life or useful life. In another example, the fluid in the fluid reservoir 310 can have a relatively high viscosity. In some embodiments, the relatively high viscosity fluid can have a higher viscosity than water or can have a viscosity that deforms or moves through the migration medium 306 when receiving a compression force as discussed herein. After the piston 231 is moved through the charging chamber 355 a first time, the migration medium membrane 412 breaks and the deflection or compression force exerted on the flexible membrane 405 deflects the fluid reservoir 310 and forces the relatively high viscosity fluid into the migration medium 306. Each movement of the piston 231 through the charging chamber 355 deflects or compresses the flexible membrane 405 and the fluid reservoir 310 and forces the relatively high viscosity fluid further through the migration medium 306 and away from the fluid reservoir 310. When the fluid, such as timer fluid, in the migration medium 306 reaches a predetermined distance from the migration medium membrane 412 or the fluid reservoir 310, the timer 130 indicates that the negative-pressure source 104 has reached an expiration of at least one of useable life or useful life.

In yet another example, the migration medium membrane 412 includes one or more apertures. The one or more apertures can each have a diameter or cross-sectional area configured to permit fluid to pass through them when the fluid in the fluid reservoir 310 reaches a predetermined pressure. When the piston 231 is moved through the charging chamber 355, the deflection or compression force exerted on the fluid reservoir 310 creates a pressure to force the fluid from the fluid reservoir 310, through the one or more apertures of the migration medium membrane 412, and into the migration medium 306. Each movement of the piston 231 through the charging chamber 355 deflects or compresses the fluid reservoir 310 and forces more fluid, from the fluid reservoir 310, through the one or more apertures of the migration medium membrane 412, and into the migration medium 306 causing the fluid in the migration medium 306 to move a distance further away from the fluid reservoir 310. When the fluid in the migration medium 306 reaches a predetermined distance from the migration medium membrane 412 or the fluid reservoir 310, the timer 130 indicates that the negative-pressure source 104 has reached an expiration of at least one of a useable life or useful life.

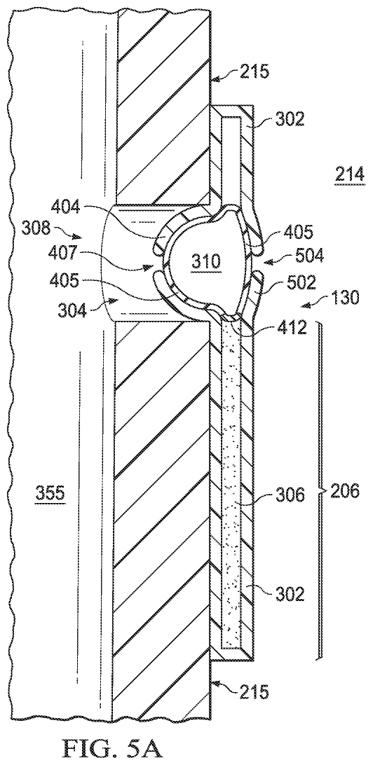

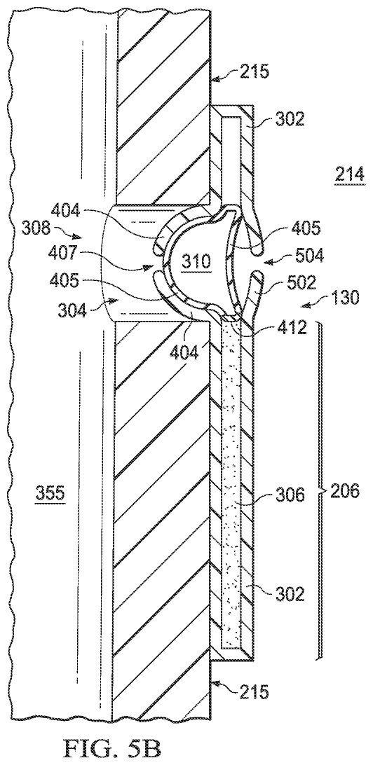

FIGS. 5A and 5B are schematic diagrams illustrating additional details that may be associated with some example embodiments of the timer 130. In the example embodiment of FIGS. 5A and 5B, the timer 130 includes the housing 302, the actuator 304, and the migration medium 306. Similar to the example embodiments illustrated in FIGS. 3, 4A, and 4B, the housing 302 may be coupled to the exterior surface of the first barrel 215. The housing 302 can be in fluid communication with both the charging chamber 355 and the ambient environment 214 surrounding the negative-pressure source 104 via the aperture 308 through the negative-pressure source 104 or the first barrel 215. The housing 302 can be configured to fit over the aperture 308 providing a seal over the aperture 308 and preventing fluid communication between the charging chamber 355 and the ambient environment 214. In the example embodiments of FIGS. 5A and 5B, the actuator 304 may additionally include a protective shell, such as a shell 502, with an aperture 504 (such as a vent) providing fluid communication between the flexible membrane 405 and the ambient environment 214. The shell 502 can be integral with or otherwise coupled to the housing 302 and enclose the fluid reservoir 310, in some embodiments.

The fluid reservoir 310 and the migration medium 306 are generally disposed in an interior of the housing 302. The fluid reservoir 310 is formed by the flexible membrane 405. The fluid reservoir 310 may contain a fluid, such as a colored fluid, configured to move through the migration medium 306. If the flexible membrane 405 and thus the fluid reservoir 310 is deflected or compressed, for example due to receiving a compression force and being pressed against the shell 404, pressure can increase in the fluid reservoir 310 and fluid in the fluid reservoir 310 can be communicated to the migration medium 306. The shell 404 in the example of FIG. 5A includes the aperture 407 to provide fluid communication between the charging chamber 355 and the flexible membrane 405 on the charging chamber side of the timer 130. The flexible membrane 405 can be coupled to an interior surface of the housing 302 to provide a seal preventing fluid communication between the ambient environment 214 and the charging chamber 355 via the aperture 407 and the aperture 504. The flexible membrane 405 can also be coupled to the interior surface of the housing 302 to permit a portion of the flexible membrane 405 to deflect or compress when receiving a compression force.

The aperture 407 and the aperture 504 are configured to permit a differential pressure to form across the actuator 304 to generate a deflection or a compression force on the fluid reservoir 310 (such as a deflection or compression force on a flexible membrane 405 containing the fluid reservoir 310). FIG. 5A illustrates an example of the fluid reservoir 310 in a non-deflected state. When the piston 231 moves toward a resting position through the charging chamber 355, a negative pressure can be created or generated within the charging chamber 355. The difference in pressure between the pressure in the charging chamber 355 and the pressure in the ambient environment 214 creates or generates a pressure differential across the fluid reservoir 310 via the aperture 407 and the aperture 504. The pressure differential across the fluid reservoir 310 deflects or creates a compression force on the fluid reservoir 310 and forces the fluid reservoir 310 against the shell 404 increasing pressure in the fluid reservoir 310. The shells 404 and 502 be sufficiently rigid to maintain its shape under the pressure differential, as illustrated in the example of FIG. 5B.

FIG. 5B illustrates an example of the fluid reservoir 310 in a deflected state. The deflection or the compression force received by the fluid reservoir 310 increases pressure in the fluid reservoir 310 and causes fluid in the fluid reservoir 310 to move into the migration medium 306. The fluid moving into and through the migration medium 306 provides an indication of a time relative to an initial movement of the piston 231 through the charging chamber 355. Thus, in some embodiments, a position of fluid within the migration medium 306 indicates a time. When the fluid reaches a distance (such as a predetermined distance) in the migration medium 306 from the migration medium membrane 412 or the fluid reservoir 310, the timer 130 provides an indication that the negative-pressure source 104 has reached at least one of an expiration of useable life or useful life.

The migration medium membrane 412 can be configured to allow fluid to communicate from the fluid reservoir 310 to the migration medium 306. After the piston 231 is moved through the charging chamber 355 a first time, the migration medium membrane 412 can break and permit a flow of fluid from the fluid reservoir 310 and into the migration medium 306 until the fluid reaches a distance from the fluid reservoir 310 indicating that the negative-pressure source 104 has reached an expiration of useable life. In some embodiments, the fluid can have a relatively high viscosity as discussed herein. After the piston 231 is moved through the charging chamber 355 a first time, the migration medium membrane 412 breaks and the deflection or compression force exerted on the fluid reservoir 310 forces the relatively high viscosity fluid into the migration medium 306. Each movement of the piston 231 through the charging chamber 355 deflects or compresses the fluid reservoir 310 and forces the relatively high viscosity fluid further through the migration medium 306 and away from the fluid reservoir 310. When the fluid in the migration medium 306 reaches a predetermined distance from the migration medium membrane 412 or the fluid reservoir 310, the timer 130 indicates that the negative-pressure source 104 has reached at least one of an expiration of useable life or useful life.

In yet another example, the migration medium membrane 412 includes one or more apertures. The one or more apertures can each have a diameter or cross-sectional area configured to permit relatively high viscosity fluid to pass through them when the relatively higher viscosity fluid in the fluid reservoir 310 reaches a predetermined pressure. When the piston 231 is moved through the charging chamber 355, the deflection or compression force exerted on the fluid reservoir 310 creates a pressure to force the relatively high viscosity fluid through the one or more apertures of the migration medium membrane 412 and into the migration medium 306. Each movement of the piston 231 through the charging chamber 355 deflects or compresses the fluid reservoir 310 and forces more relatively high viscosity fluid through the one or more apertures of the migration medium membrane 412 and into the migration medium 306 causing the relatively high viscosity fluid in the migration medium 306 to move a distance further away from the fluid reservoir 310. When the fluid in the migration medium 306 reaches a predetermined distance from the migration medium membrane 412 or the fluid reservoir 310, the timer 130 indicates that the negative-pressure source 104 has reached at least one of an expiration of useable life or useful life.

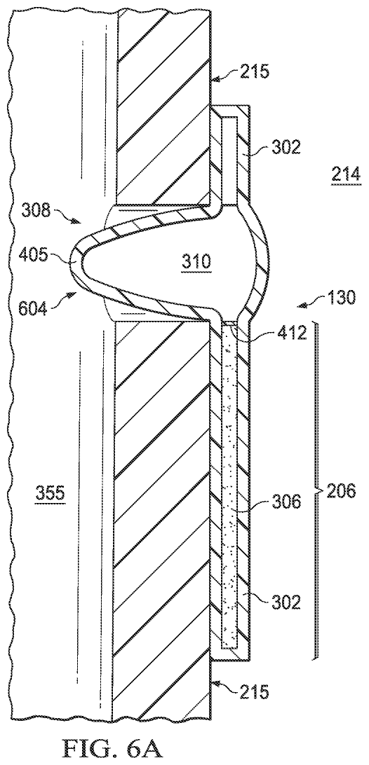

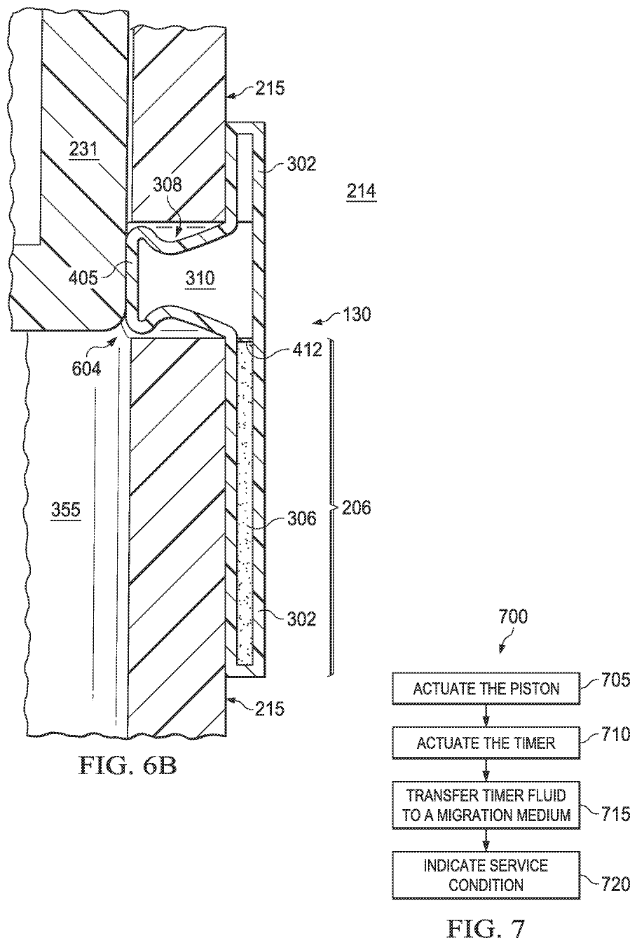

FIGS. 6A and 6B are schematic diagrams illustrating additional details that may be associated with some example embodiments of the timer 130. In the example embodiment of FIGS. 6A and 6B, the timer 130 includes the housing 302, an actuator 604, and the migration medium 306. Similar to the example embodiments illustrated in FIGS. 3, 4A, 4B, 5A, and 5B. the housing 302 may be coupled to the exterior surface of the first barrel 215. The housing 302 can be in fluid communication with both the charging chamber 355 and the ambient environment 214 surrounding the negative-pressure source 104 via the aperture 308 through the negative-pressure source 104 or the first barrel 215. The housing 302 can be configured to fit over the aperture 308 providing a seal over the aperture 308 and preventing fluid communication between the charging chamber 355 and the ambient environment 214. In the example embodiments of FIGS. 6A and 6B, the actuator 604 can extend into the charging chamber 355 and configured to allow the piston 231 to engage and deflect the actuator 604. The actuator 604 can be integral with or otherwise coupled to the housing 302 and enclose the fluid reservoir 310 formed by walls 311 in some embodiments, as discussed herein.

The fluid reservoir 310 and the migration medium 306 are generally disposed in an interior of the housing 302. The fluid reservoir 310 is formed by walls 311. The fluid reservoir 310 may contain a fluid, such as a colored fluid, configured to move through the migration medium 306. If the fluid reservoir 310 is deflected or compressed, for example due to receiving a compression force, fluid in the fluid reservoir 310 can be communicated to the migration medium 306.

In some embodiments, the actuator 604 that extends into the charging chamber 355 and is configured to engage the piston 231 as the piston 231 moves through the charging chamber 355. The fluid reservoir 310 can be positioned inside the actuator 604 as discussed and illustrated herein to permit a portion of the fluid reservoir 310 to deflect or compress when receiving a compression force. For example, one or more walls 311 of the fluid reservoir 310 can be flexible.

The actuator 604 is configured to receive contact from the piston 231 as the piston 231 move through the charging chamber 355. Contact from the piston 231 as the piston 231 moves through the charging chamber 355 can generate a deflection or a compression force on the actuator 604 and the fluid reservoir 310. FIG. 6A illustrates an example of the actuator 604 and the fluid reservoir 310 in a non-deflected state. As shown in FIG. 6A, the actuator 604 extends into the charging chamber 355 so that when the piston 231 moves toward a resting position through the charging chamber 355, the piston 231 can make contact with the actuator 604 and deflect the actuator 604 and the fluid reservoir 310. The deflection of the actuator 604 and the fluid reservoir 310 reduces the volume of the fluid reservoir 310 and increases the pressure within the fluid reservoir 310.

FIG. 6B illustrates an example of the piston 231 making contact with and deflecting the actuator 604 and the fluid reservoir 310. The deflection or the compression force received by the actuator 604 and the fluid reservoir 310 can cause fluid in the fluid reservoir 310 to move into the migration medium 306. The fluid moving into and through the migration medium 306 provides an indication of a time relative to an initial movement of the piston 231 through the charging chamber 355. Thus, in some embodiments, a position of fluid within the migration medium 306 indicates a time. When the fluid reaches a distance (such as a predetermined distance) in the migration medium 306 from the migration medium membrane 412 or the fluid reservoir 310, the timer 130 provides an indication that the negative-pressure source 104 has reached at least one of an expiration of useable life or useful life.

The migration medium membrane 412 can be configured to prevent fluid from communicating from the fluid reservoir 310 to the migration medium 306. After the piston 231 is moved through the charging chamber 355 a first time, the migration medium membrane 412 can break and permit a flow of fluid from the fluid reservoir 310 and into the migration medium 306 until the fluid reaches a distance from the fluid reservoir 310 indicating that the negative-pressure source 104 has reached an expiration of useable life. In some embodiments, the fluid can have a relatively high viscosity as discussed herein. After the piston 231 is moved through the charging chamber 355 a first time, the migration medium membrane 412 breaks and the deflection or compression force exerted on the fluid reservoir 310 forces the relatively high viscosity fluid into the migration medium 306. Each movement of the piston 231 through the charging chamber 355 deflects or compresses the fluid reservoir 310 and forces the relatively high viscosity fluid further through the migration medium 306 and away from the fluid reservoir 310. When the fluid in the migration medium 306 reaches a predetermined distance from the migration medium membrane 412 or the fluid reservoir 310, the timer 130 indicates that the negative-pressure source 104 has reached at least one of an expiration of useable life or useful life.

FIG. 7 is a flow diagram illustrating an example method 700 to operate a negative-pressure source, such as the negative-pressure source 104. In the example of FIG. 7, a piston can be actuated at step 705. In some embodiments, for example, the piston 231 of the negative-pressure source 104 can be actuated to move it through the charging chamber 355. At step 710, a timer, such as the timer 130, can be actuated in response to actuation of the piston. In some embodiments, the timer can be actuated by direct or indirect contact with the piston. In other embodiments, the timer can be pneumatically actuated. For example, the timer 130 can be pneumatically actuated by a pressure differential resulting from operation of a piston in the negative-pressure source 104. At step 715, a timer fluid can be transferred to a migration medium, such as the migration medium 306. For example, actuating the timer at step 710 can compress the fluid reservoir 310 to engage the timer fluid with the migration medium. At step 715, a service condition can be indicated. For example, in some embodiments, the service condition can be indicated by timer fluid visible through a window, such as the view port 206.

As a further example, as illustrated in any of FIGS. 1-7, an apparatus for negative pressure therapy, such as a negative-pressure source 104, can include a negative-pressure chamber, such as a charging chamber 355. The apparatus can also include a migration medium. The apparatus can further include a reservoir, such as the fluid reservoir 310, having a timer fluid. The reservoir can be configured to transfer the timer fluid to the migration medium in response to a pressure differential between the negative-pressure chamber and an ambient pressure, such as the pressure in an ambient environment 214. The reservoir can include one or more walls (such as walls 311) in which the timer fluid can be disposed. A membrane (such as flexible membrane 405) can be configured to collapse in response to the pressure differential to compress the reservoir. The reservoir can include a vent, such as the second housing aperture 504b, between the cavity and the ambient environment 214. The reservoir can include a port, such as the first housing aperture 504a, between the reservoir and the negative-pressure chamber. The membrane can also be disposed in the cavity and can be configured to collapse in response to the pressure differential. The apparatus can also include a sacrificial seal (such as the migration medium membrane 412) between the migration medium and the reservoir. The timer fluid can be configured to break the sacrificial seal in response to the pressure differential. The timer fluid can migrate through the migration medium at a predetermined rate and for a predetermined period of time. In some embodiments, the predetermined period can be indicative of a useful life for the apparatus.

The systems, apparatuses, and methods described herein may provide significant advantages. For example, an operator using a negative-pressure source that includes a service timer such as the timer 130 can automatically provide an indication when the negative-pressure source has a high probability of being contaminated or has a high probability of having less than optimal or adequate functionality. In other words, an operator does not have to remember the first time a negative-pressure source was used or a number of times that a negative-pressure source has been used to determine whether the negative-pressure source is in a condition for safe and effective use.

While shown in a few illustrative embodiments, a person having ordinary skill in the art will recognize that the systems, apparatuses, and methods described herein are susceptible to various changes and modifications. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 102, the container 112, or both may be eliminated or separated from other components for manufacture or sale. In other example configurations, the controller 110 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described or illustrated in the context of some example embodiments may also be omitted or combined with features, elements, and aspects of other example embodiments. Features, elements, and aspects described herein may also be combined or replaced by alternative features serving the same, equivalent, or similar purpose without departing from the scope of the invention defined by the appended claims.

1. An apparatus for providing negative-pressure therapy, the apparatus comprising:
a negative-pressure chamber;
a piston disposed in the negative-pressure chamber and configured to move along an axis of the negative-pressure chamber;
a timer; and
an actuator configured to initiate the timer in response to movement of the piston along the axis of the negative-pressure chamber.

2. The apparatus of claim 1, wherein the timer is configured to be initiated in response to a differential pressure across the timer generated by the movement of the piston along the axis of the negative-pressure chamber.

3. The apparatus of claim 1, wherein the actuator is configured to initiate the timer in response to an engagement between the piston and the actuator.

4. The apparatus of claim 1, wherein the apparatus is a manually-actuated negative-pressure pump.

5. The apparatus of claim 1, wherein the timer is configured to provide an indication that a usable life of the apparatus has expired when the timer reaches an expiration time.

6. The apparatus of claim 1, wherein the timer comprises:
a housing comprising:
a first vent configured to provide fluid communication between an interior of the housing and an ambient environment;
a second vent configured to provide fluid communication between the interior of the housing and the negative-pressure chamber;
a flexible membrane disposed in the interior of the housing, wherein the flexible membrane forms a fluid reservoir; and
a migration medium disposed in the interior of the housing and configured to receive fluid from the fluid reservoir in response to a compression of the flexible membrane, wherein the migration medium begins receiving fluid from the fluid reservoir when the timer is initiated.

7. The negative-pressure pump of claim 6, wherein the second vent is configured to provide fluid communication between the interior of the housing and the negative-pressure chamber via an aperture in a negative-pressure chamber wall forming the negative-pressure chamber.

8. The negative-pressure pump of claim 6, wherein a differential pressure across the fluid reservoir generates the compression of the fluid reservoir.

9. The negative-pressure pump of claim 6, wherein the housing further comprises a view port configured to permit viewing of the fluid in the migration medium.

10. A negative-pressure pump comprising:
a charging chamber;
a piston disposed in the charging chamber and configured to move along an axis of the charging chamber; and
a timer configured to be initiated in response to receiving a force and to indicate a service condition of the negative-pressure pump, the timer comprising:
a flexible membrane forming a fluid reservoir, and
a migration medium configured to receive fluid from the fluid reservoir in response to a compression of the fluid reservoir, wherein the migration medium begins receiving fluid from the fluid reservoir when the timer is initiated.

11. The negative-pressure pump of claim 10, wherein the service condition corresponds to an end of a usable life of the negative-pressure pump.

12. The negative-pressure pump of claim 10, wherein the force is generated in response to a differential pressure across the timer generated by movement of the piston along the axis of the charging chamber.

13. The negative-pressure pump of claim 10, wherein the force is an external compression force.

14. The negative-pressure pump of claim 10, wherein the force is generated in response to an engagement between the piston and the timer.

15. The negative-pressure pump of claim 10, wherein the piston is configured to be actuated manually.

16. (canceled)

17. The negative-pressure pump of claim 10, wherein a differential pressure across the fluid reservoir creates the compression of the fluid reservoir.

18. The negative-pressure pump of claim 10, wherein the timer further comprises a view port configured to permit viewing of the fluid in the migration medium.

19.-42. (canceled)

43. A negative-pressure pump comprising:
a charging chamber;
a timer configured to indicate a service condition of the negative-pressure pump; and
a piston disposed in the charging chamber and configured to move along an axis of the charging chamber to actuate the timer.

44. The negative-pressure pump of claim 43, wherein the service condition is an end of a usable life of the negative-pressure pump.

45. The negative-pressure pump of claim 43, wherein the piston is configured to generate a differential pressure across the timer to actuate the timer.

46. The negative-pressure pump of claim 43, wherein the timer is configured to be actuated based on an engagement between the piston and the timer.

47. The negative-pressure pump of claim 43, wherein the piston is configured to be actuated manually.

48. The negative-pressure pump of claim 43, wherein the timer comprises:
a flexible membrane forming a fluid reservoir; and
a migration medium configured to receive fluid from the fluid reservoir in response to a compression of the flexible membrane, wherein the migration medium begins receiving fluid from the fluid reservoir when the timer is initiated.

49. The negative-pressure pump of claim 48, wherein a differential pressure across the flexible membrane creates the compression of the flexible membrane.

50. The negative-pressure pump of claim 48, wherein the timer further comprises a view port configured to permit viewing of the fluid in the migration medium.

51.-54. (canceled)
